(19) United States
(12) Patent Application Publication
(10) Pub. No.: US 2022/0304916 A1
(43) Pub. Date: Sep. 29, 2022

(54) MODIFIED BLACK SOLDIER FLY LARVAE OIL WITH MODIFIED LAURIC ACID FOR TREATMENT AGAINST BIOFILM FORMATION AND MICROORGANISM GROWTH

(71) Applicant: BIO-BEE SDE ELIYAHU LTD, Sde Eliyahu (IL)

(72) Inventors: Hadas RICHTER, Beit Elazari (IL); Shimon STEINBERG, Sde Eliyahu (IL); Yuval BARON, Sde Eliyahu (IL); Amit INBART, Beit Elazari (IL)

(73) Assignee: BIO-BEE SDE ELIYAHU LTD, Sde Eliyahu (IL)

(21) Appl. No.: 17/613,522

(22) PCT Filed: May 21, 2020
(86) PCT No.: PCT/IL2020/050560

§ 371 Date: Nov. 23, 2021

Related U.S. Application Data
(60) Provisional application No. 62/851,942, filed on May 23, 2019.

(51) Int. Cl. A61K 8/92; A61P 31/04; A61P 17/00; A61Q 17/00; C11B 1/10; C12N 9/20

(52) U.S. Cl. CPC A61K 8/927; A61P 31/04; A61P 17/00; A61Q 17/005; C11B 1/10; C12N 9/20

(57) ABSTRACT

Methods, formulations and production systems are provided. Methods comprise extracting black soldier fly larvae (BSFL) oil by processing BSFL, modifying the BSFL oil into modified BSFL (MBSFL) oil by converting triglycerides in the BSFL oil to medium chain fatty acids (MCFAs) in the form of monoglycerides, fatty acid salts and/or free fatty acids, e.g., by saponification and/or hydrolysis, and applying the MBSFL oil to suppress biofilm development and/or microorganism growth (e.g., of Gram-positive and/or Gram-negative bacteria, fungi and possibly viruses). Applications of the MBSFL oil include dermal and/or oral applications, topical therapy, as well as applications to medical equipment and industrial applications.

### BACKGROUND OF THE INVENTION

### 1. Technical Field

The present invention relates to the field of treatments against biofilm formation and fungal and bacterial growth, and more particularly, to utilizing modified black soldier fly larvae (MBSFL) oil with released medium chain fatty acids (MCFAs), mainly lauric acid, in the form of laurate (e.g., sodium laurate, potassium laurate etc.), monolaurin and/or free lauric acid.

### 2. Discussion of Related Art

Black soldier fly (Hermetia illucens) larvae (BSFL) are rich in fat, with levels ranging between 15% and 49% on dry matter basis. Notably, the fatty acid profile of the prepupae is high in the medium-chain fatty acids (MCFAs), with lauric acid (C12:0) being the major component. Lauric acid is known to have profound antiviral, antifungal and antibacterial activity, and particularly to be active against Gram positive bacteria. The fatty acid profile of the prepupae contains additional MCFAs such as capric acid (C10:0) and caprylic acid (C8:0). Trials showed that black soldier fly prepupal fat (0.58 g C12:0/100 ml) suppressed growth of Lactobacilli, with the most substantial antibacterial effects against D-streptococci infections in pigs. In in vivo conditions it has been suggested that these positive effects are most likely seen when farming conditions and/or health status are sub-optimal (Gasco et al. 2018, Can diets containing insects promote animal health? Journal of Insects as Food and Feed 4(1): 1-4, incorporated herein by reference in its entirety).

It has also been reported that whereas medium-chain fatty acids (MCFAs), monoglycerides and free fatty acids (FFA) sourced from virgin coconut oil (VCO) exhibit antimicrobial activity, the triglycerides and diglycerides in the oil were shown to have a lesser antimicrobial activity. It has been suggested that VCO may be metabolized to release its component MCFAs, caprylic acid (C8:0), capric acid (C10:0), and lauric acid (C12:0) to exert its antimicrobial effects (Shilling et al. 2013, Antimicrobial effects of virgin coconut oil and its medium-chain fatty acids on Clostridium difficile; Journal Of Medicinal Food 16 (12), 1079-1085, incorporated herein by reference in its entirety).

Lauric acid in insect larvae is stored mainly as triglycerides (Liland et al. 2017, Modulation of nutrient composition of black soldier fly (Hermetia illucens) larvae by feeding seaweed-enriched media, PLoS ONE 12(8): e0183188, incorporated herein by reference in its entirety). There are several ways known in the industry to break triglycerides into monoglycerides and free fatty acids to enhance its antimicrobial properties. For example, in WIPO Publication No. 2007067028, incorporated herein by reference in its entirety, coconut oil and palm kernel were hydrolyzed using a catalytic activity of 1,3 positional specific lipases. The modified oil compositions comprised of free fatty acids (>9.4%), monoglycerides (>1.3%), diglycerides (>22.8%) and triglycerides (>25%), were able to inhibit the growth of Gram-positive bacteria, e.g., Staphylococcus aurous aureus, Listeria monocytogenes, Sterptococcus pyogene, Gram-negative bacteria, e.g., Vibrio cholerae, Escherichia coli and yeast, e.g., Candida albicans.

Fatty acids and monoglycerides produce their killing/inactivating effects by several mechanisms. An early postulated mechanism was the perturbing of the plasma membrane lipid bilayer. The antiviral action attributed to monolaurin is that of fluidizing the lipids and phospholipids in the envelope of the virus, causing the disintegration of the microbial membrane. More recent studies indicate that one antimicrobial effect in bacteria is related to monolaurin's interference with signal transduction/toxin formation (Projan, et al. 1994, Glycerol monolaurate inhibits the production of Blactamase, toxic shock syndrome toxin-1, and other staphylococcal exoproteins by interfering with signal transduction. J Bacteriology 176:4204-4209, incorporated herein by reference in its entirety). Another antimicrobial effect in viruses is due to lauric acid's interference with virus assembly and viral maturation (Hornung et al. 1994, Lauric acid inhibits the maturation of vesicular stomatitis virus; Journal of General Virology 75 (Pt 2)(2):353-61, incorporated herein by reference in its entirety). The third mode of action may be on the immune system itself (Witcher et al. 1996, Modulation of immune cell proliferation by glycerol monolaurate. Clinical and Diagnostic Laboratory Immunology 3:10-13, incorporated herein by reference in its entirety).

Hess et al. 2015, The natural surfactant glycerol monolaurate significantly reduces development of Staphylococcus aureus and Enterococcus faecalis biofilms, Surgical Infections 16(5): 538-542, incorporated herein by reference in its entirety, teaches using the natural surfactant glycerol monolaurate (GML) to inhibit biofilm development.

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides a method comprising extracting black soldier fly larvae (BSFL) oil by processing BSFL, modifying the BSFL oil into modified BSFL (MBSFL) oil by converting triglycerides in the BSFL oil to monoglycerides, fatty acid salts and/or free fatty acids (FFA) of medium chain fatty acids (MCFAs), and applying the MBSFL oil to suppress biofilm development and/or microorganism growth.

One aspect of the present invention provides a system comprising a processing unit configured to extract black soldier fly larvae (BSFL) oil from BSFL, a conversion unit configured to convert triglycerides in the prepared BSFL oil into monoglycerides, fatty acid salts and/or FFA of medium chain fatty acids (MCFAs) to yield modified black soldier fly larvae (MBSFL) oil, and a formulation unit configured to prepare from the MBSFL oil a formulation that suppresses biofilm development and/or microorganism growth.

One aspect of the present invention provides a topical dermal or oral composition, comprising modified black soldier fly larvae (MBSFL) oil, modified from BSFL oil extracted from processed BSFL and comprising monoglycerides, fatty acid salts and/or FFA of medium chain fatty acids (MCFAs) converted from triglycerides in the BSFL oil.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIG. 1 is a high-level schematic flowchart illustration of a method, according to some embodiments of the invention.

FIG. 2 is a high-level schematic illustration of a system, according to some embodiments of the invention.

FIG. 3 provides results indicating the effect of BSFL oil on Pseudomonas aeruginosa (PAO1) growth in planktonic environment, according to some embodiments of the invention.

FIGS. 4A-F provide comparative results indicating the effect of MBSFL oil and lauric acid (LA) on the growth of various types of microorganisms in planktonic environment, specifically Pseudomonas aeruginosa (PAO1, FIG. 4A), Staphylococcus aureus (SA, FIG. 4B), Streptococcus mutans (SM, FIG. 4C), Lactobacillus (L, FIG. 4D), Candida albicans (CA, FIG. 4E) and Candida glabrata (CG, FIG. 4F), according to some embodiments of the invention.

FIG. 5 provides results indicating the effect of BSFL oil on biofilm formation of Staphylococcus aureus (SA), according to some embodiments of the invention.

FIGS. 6A-E provide comparative results indicating the effect of MBSFL oil and lauric acid (LA) on biofilm formation of various types of microorganisms: Pseudomonas aeruginosa (PAO1, FIG. 6A), Staphylococcus aureus (SA, FIG. 6B), Streptococcus mutans (SM, FIG. 6C), Candida albicans (CA, FIG. 6D) and Candida glabrata (CG, FIG. 6E), according to some embodiments of the invention.

FIGS. 7A-F provide comparative results indicating the effect of chlorhexidine acetate (CHX) on planktonic growth and biofilm formation of various types of microorganisms: Pseudomonas aeruginosa (PAO1, FIG. 7A), Staphylococcus aureus (SA, FIG. 7B), Streptococcus mutans (SM, FIG. 7C), Lactobacillus (L, FIG. 7D), Candida albicans (CA, FIG. 7E) and Candida glabrata (CG, FIG. 7F), according to some embodiments of the invention.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein are for the purpose of description and should not be regarded as limiting.

Embodiments of the present invention provide efficient and economical methods and mechanisms for modifying BSFL oil to enhance medium chain fatty acids (MCFAs) in the form of monoglycerides, fatty acid salts and/or FFA, and thereby provide improvements to the technological field of treatments against biofilm formation and fungal, viral and bacterial growth. Methods, formulations and production systems are provided. Methods comprise extracting black soldier fly larvae (BSFL) oil by processing BSFL, modifying the BSFL oil into modified BSFL (MBSFL) oil by converting triglycerides in the BSFL oil to monoglycerides, fatty acid salts and/or FFA of MCFAs, e.g., by saponification and/or hydrolysis, and applying the MBSFL oil to suppress biofilm development and/or microorganism growth (e.g., of Gram-positive and/or Gram-negative bacteria, fungi and possibly viruses). Applications of the MBSFL oil include dermal, topical and/or oral applications, as well as applications to medical equipment and industrial pipework.

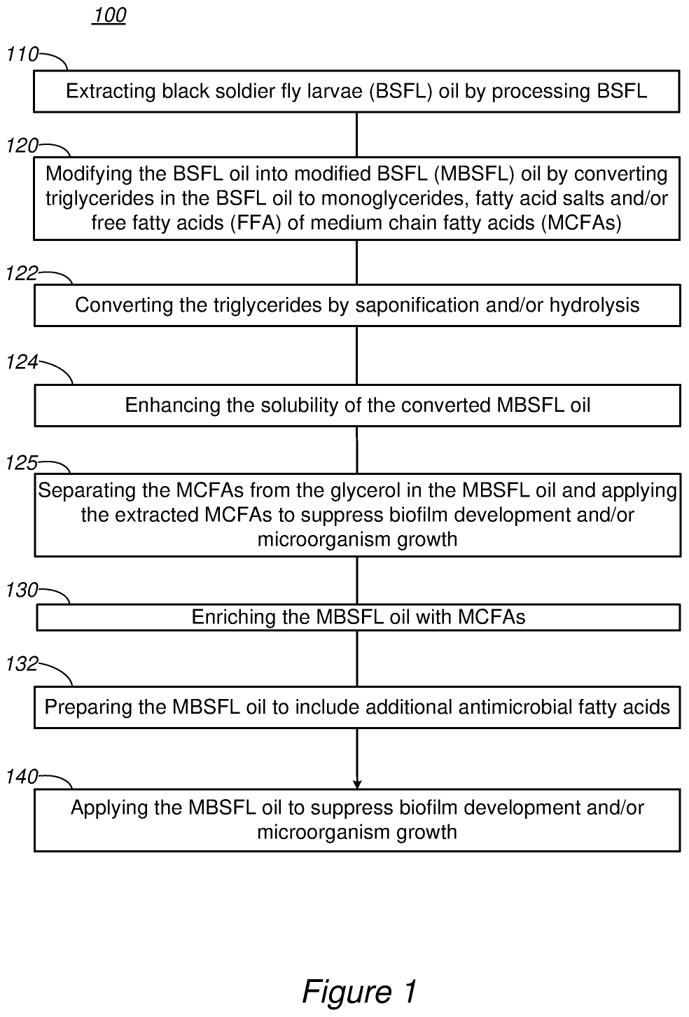

FIG. 1 is a high-level schematic flowchart illustration of a method 100, according to some embodiments of the invention. Method 100 may comprise the following stages, irrespective of their order. Method 100 comprises extracting black soldier fly larvae (BSFL) oil by processing BSFL (stage 110), modifying the BSFL oil into modified BSFL (MBSFL) oil by converting triglycerides in the BSFL oil to monoglycerides, fatty acid salts and/or FFA of MCFAs (stage 120), and applying the MBSFL oil to suppress biofilm development and/or microorganism growth (stage 140), e.g., of bacteria, fungi, viruses and/or protozoa. In various embodiments, BSFL oil extraction 110 may precede BSFL oil conversion 120 and/or conversion of triglycerides 120 in the BSFL may precede BSFL oil extraction 110, in which case the extracted BSFL oil may already be enriched with MCFAs.

It is noted that the MCFAs may comprise mainly lauric acid, for example in the form of laurate, e.g., sodium laurate, potassium laurate etc., monolaurin and/or free lauric acid.

It is further noted that typically BSFL oil comprises clear fat (e.g., having >99% fat), that contains ca. 45% lauric acid (as a non-limiting example) in the form of triglycerides, which are modified in MBSFL oil to the form of laurate, e.g., sodium laurate, potassium laurate etc., monolaurin and/or free lauric acid. The extraction process of the oil from the BSFL leaves behind the rest of the biomass of the larvae, termed “BSF meal”, “BSFL meal” or “protein meal”, which contains only 5-15% oil, which, if converted, may comprise ca. 40% lauric acid in the form of laurate, e.g., sodium laurate, potassium laurate etc., monolaurin and/or free lauric acid. In various embodiments, different oil levels may be produced in both BSFL/MBSFL oils and BSFL meal, and, in case the latter comprises a large oil portion, it may too be converted into monoglycerides, fatty acid salts and/or FFA of MCFAs for various applications (see an example below).

In certain embodiments, converting triglycerides in the BSFL oil to monoglycerides, fatty acid salts and/or FFA of MCFAs 120 may be carried out by saponification and/or hydrolysis (stage 122). Hydrolysis may comprise acidic and/or enzymatic hydrolysis. In certain embodiments, method 100 may comprise enhancing the solubility of the converted MBSFL oil with respect to the BSFL oil (stage 124), as disclosed below. Conversion 120 may comprise breaking off fatty acids from triglyceride fats in the BSFL oil to form monoglycerides and/or free fatty acids and/or fatty acid salts of MCFAs to exert the antimicrobial and/or antibiofilm activity.

In certain embodiments, method 100 may further comprise separating the MCFAs from the glycerol in the MBSFL oil and applying the extracted MCFAs to suppress biofilm development and/or microorganism growth (stage 125). In certain embodiments, method 100 may comprise enriching the MBSFL oil with MCFAs (stage 130), possibly separated from MBSFL oil. In certain embodiments, method 100 may comprise preparing the MBSFL oil to include additional antimicrobial fatty acids (stage 132), e.g., preparing the MBSFL oil to comprise a combination of MCFAs lauric acid (C12:0) and capric acid (C10:0) with optional antimicrobial fatty acids myristic acid (C14:0), palmitoleic acid (C16:1), oleic acid (C18:1), linoleic acid (C18:2) and/or alpha linolenic acid (C18:3), converted by saponification and/or hydrolysis from the respective fatty acid triglycerides—to yield antimicrobial and antibiofilm properties.

Method 100 may comprise applying the MBSFL oil to suppress biofilm development and/or microorganism growth in a wide range of applications. In various embodiments, applying the MBSFL oil 140 may be carried out against any of Gram-positive bacterial biofilms, Gram-negative bacterial biofilms, fungal biofilms and/or growth of micro-organisms such as fungi, protozoa, bacteria and/or viruses. In various embodiments, applying the MBSFL oil 140 may be carried out in any of dermal, topical and/or oral applications (see examples below); for disinfecting medical equipment such as catheters; and/or in industrial applications such as disinfecting pipes.

Certain embodiments comprise compositions or formulations comprising MBSFL oil, modified from BSFL oil extracted from processed BSFL and comprising monoglycerides, fatty acid salts and/or FFA of MCFAs converted from triglycerides in the BSFL oil. In certain embodiments, the compositions or formulations may comprise topical dermal and/or oral formulations. In certain embodiments, the compositions or formulations may comprise compositions for disinfecting medical equipment such as catheters and/or industrial pipework.

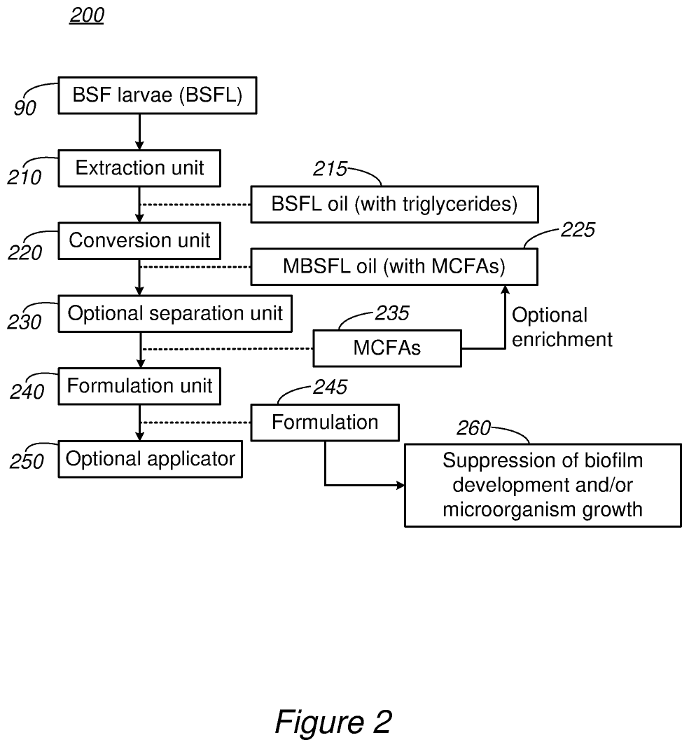

FIG. 2 is a high-level schematic illustration of a system 200, according to some embodiments of the invention. System 200 comprises an extraction unit 210 configured to extract BSFL oil 215 from BSFL 90, a conversion unit 220 configured to convert triglycerides in prepared BSFL oil 215 into monoglycerides, fatty acid salts and/or FFA of MCFAs—to yield modified black soldier fly larvae (MBSFL) oil 225, and a formulation unit 240 configured to prepare from MBSFL oil 225 a formulation 245 that suppresses biofilm development and/or microorganism growth 260, e.g., of bacteria, fungi, viruses and/or protozoa. It is noted that the MCFAs may comprise mainly lauric acid, for example in the form of laurate, e.g., sodium laurate, potassium laurate etc., monolaurin and/or free lauric acid.

In an experimental, non-limiting example, BSFL 90 were used as raw material, after drying to a moisture content of less than 3%, to extract oil therefrom using a mechanical press (expeller). In certain embodiments, solvent extraction (e.g., using hexane, petroleum ether, water) or other technologies may be applied to process BSFL 90. MBSFL oil 225 (converted from BSFL oil 215), was prepared using a saponification process (by which triglycerides are reacted with sodium or potassium hydroxide to produce glycerol and a fatty acid salt). BSFL oil 215 was heated to 60-70° C. KOH was mixed with bi-distilled water at a 1:3 ratio accordingly, and then mixed with heated BSFL oil 215 in a ratio according to its saponification number, which was set to 19.9% (2500 g BSFL oil, 1500 g bi-distilled H2O, 497.5 g KOH analytical). In another example, a similar mixture was prepared, but with an excess amount of KOH (5% more than indicated according to the saponification number). In the data below resulting MBSFL oil 225 (indicated as lot 0030.111217-181125) compared to the source BSFL oil 215 (indicated as lot 0030.111217). Once a homogenous blend was reached, it was then mixed with an electric hand mixer for 20 seconds every one minute, until a viscous mixture was obtained. The mixture was then transferred to a double jacket heated pot, and was heated followed by a gentle mixing every 20 minutes. The end of the heating process was set to 15% decrease in water content (which took approximately three hours).

Table 1 provides results of triacylglycerols (TAG), diacylglycerols (DAG) and monoacylglycerols (MAG) composition, and Free Fatty Acids (FFA) for BSFL oil and converted, MBSFL oil. BSFL oil glycerides composition contains mainly TAG, with no DAG or MAG detected. The saponification process increased MAG composition in the sample to 7.3 (g/100 g Oil), dominated by the dodecanoic (lauric) acid monoglyceride (2.39 g/100 g Oil).

TABLE 1
TAG, DAG and MAG composition and FFA content in BSFL oil before and after conversion
(test method for DAG, MAG composition: MP 0118 REV3 2012; Test method for
TAG composition: REG CE 273/2008; Test method for FFA: AOCS Ca 5a-40).

| | BSFL Oil | MBSFL Oil |
|---|---|---|
| Component | (lot 0030.111217) | (lot 0030.111217-181125) |
| FFA (% oleic acid) | 0.85 ± 0.05 | 3.13 |
| Triglyceride C28 (%) | 0.05 ± 0.01 | Not determinable |
| Triglyceride C30 (%) | 0.07 ± 0.01 | Not determinable |
| Triglyceride C32 (%) | 0.16 ± 0.01 | Not determinable |
| Triglyceride C34 (%) | 1.72 ± 0.02 | Not determinable |
| Triglyceride C36 (%) | 21.56 ± 0.08 | Not determinable |
| Triglyceride C38 (%) | 12.47 ± 0.07 | Not determinable |
| Triglyceride C40 (%) | 10.61 ± 0.03 | Not determinable |
| Triglyceride C42 (%) | 8.14 ± 0.03 | Not determinable |
| Triglyceride C44 (%) | 5.90 ± 0.03 | Not determinable |
| Triglyceride C46 (%) | 12.00 ± 0.06 | Not determinable |
| Triglyceride C48 (%) | 12.43 ± 0.09 | Not determinable |
| Triglyceride C50 (%) | 6.10 ± 0.03 | Not determinable |
| Triglyceride C52 (%) | 5.65 ± 0.04 | Not determinable |
| Triglyceride C54 (%) | 3.14 ± 0.03 | Not determinable |
| Monolaurin (g/100 g Oil) | Not determinable | 2.39 ± 0.16 |
| Monomyristin (g/100 g Oil) | Not determinable | 0.59 ± 0.08 |
| Monopalmitin (g/100 g Oil) | Not determinable | 1.21 ± 0.09 |
| Monopalmitolein (g/100 g Oil) | Not determinable | 0.21 ± 0.07 |
| Monostearin (g/100 g Oil) | traces | 0.97 ± 0.09 |
| Monoolein (g/100 g Oil) | Not determinable | 1.02 ± 0.09 |
| Monolinolein (g/100 g Oil) | Not determinable | 0.94 ± 0.09 |
| 1,2-Distearin (g/100 g Oil) | Not determinable | Not determinable |
| 1,2-Diolein (g/100 g Oil) | Not determinable | Not determinable |
| 1,2-Dipalmitin (g/100 g Oil) | Not determinable | Not determinable |

Table 2 provides data of the fatty acids profile of the MBSFL oil. Lauric acid, C12:0, is the major component of medium-chain fatty acids (MCFAs) found in the MBSFL oil (45.48 g/100 g oil), however, MBSFL oil contains additional MCFA capric acid, C10:0 (1.03 g/100 g oil). MBSFL oil contains additional fatty acids such as n-6 linoleic acid, C18:2 (10.7603 g/100 g oil), n-7 palmitoleic acid, C16:1 (3.09 g/100 g oil) and n-9 oleic acid, C18:1 (12.5303 g/100 g oil). These fatty acids were found to exert significant antimicrobial activity against various microorganisms such as oral pathogens Streptococcus mutans, Candida albicans, Aggregatibacter actinomycetemcomitans, Fusobacterium nucleatum, and Porphyromonas gingivalis (Huang 2010, Antimicrobial activity of n-6, n-7 and n-9 fatty acids and their esters for oral microorganisms, Arch Oral Biol, 55(8): 555-560; Dilika et al. 2000, Antibacterial activity of linoleic and oleic acids isolated from Helichrysum pedunculatum: a plant used during circumcision rites; Fitoterapia, 71(4): 450-452). MBSFL oil contains 0.77 g/100 g oil n-3 alpha linolenic acid (C18:3). In a study it was demonstrated that alpha linolenic acid and its ester derivatives exhibited strong antibacterial activity against various oral pathogens, including S. mutans, C. albicans, A. actinomycetemcomitans, F. nucleatum, and P. gingivalis (Huang et al. 2010, A novel bioactivity of omega-3 polyunsaturated fatty acids and their ester derivatives; Molecular Oral Biology 25(1):75-80). MBSFL oil contains also 10.19 g/100 g oil myristic acid (C14:0) which was shown to inhibit the growth of the Gram positive bacterium, L. monocytogenes (Chen et al. 2019; Antimicrobial potential of myristic acid against Listeria monocytogenes in milk; The Journal of Antibiotics 72:298-305). Therefore, it is possible that the combination of MCFAs lauric acid (C12:0) and capric acid (C10:0) with potential antimicrobial fatty acids myristic acid (C14:0), palmitoleic acid (C16:1), oleic acid (C18:1), linoleic acid (C18:2) and/or alpha linolenic acid (C18:3) also composing the MBSFL oil and undergo the conversion process (saponification) contributes to the overall antimicrobial and antibiofilm properties of the MBSFL oil.

TABLE 2
Fatty acid profile of BSFL oil before and after conversion (MBSFL oil).
Test methods: EN 12966-2, EN 12966-1 and EN 12966-4.

| | BSFL Oil | MBSFL Oil |
|---|---|---|
| Component | (lot 0030.111217) | (lot 0030.111217-181125) |
| Caprylic acid (C 8:0, g/100 g oil) | 0.01 ± 0.01 | Not determinable |
| Capric acid (C 10:0, g/100 g oil) | 1.07 ± 0.05 | 1.03 |
| Lauric acid (C 12:0, g/100 g oil) | 42.80 ± 3.13 | 45.48 |
| Myristic acid (C 14:0, g/100 g oil) | 10.72 ± 0.76 | 10.19 |
| Palmitic acid (C 16:0, g/100 g oil) | 14.07 ± 1.06 | 13.22 |
| Palmitoleic acid (C 16:1, g/100 g oil) | 3.12 ± 0.11 | 3.09 |
| Stearic acid (C 18:0, g/100 g oil) | 3.13 ± 0.27 | 2.94 |
| Oleic acid (C 18:1, g/100 g oil) | 11.47 ± 0.14 | 12.53 |
| Linoleic acid (C 18:2, g/100 g oil) | 11.57 ± 0.58 | 10.76 |
| Alpha linolenic acid (C 18:3, g/100 g oil) | 0.78 ± 0.01 | 0.77 |

In certain embodiments, MBSFL oil 225 may comprise a combination of MCFAs lauric acid (C12:0) and capric acid (C10:0) with optional antimicrobial fatty acids myristic acid (C14:0), palmitoleic acid (C16:1), oleic acid (C18:1), linoleic acid (C18:2) and/or alpha linolenic acid (C18:3), converted by saponification and/or hydrolysis from the respective fatty acid triglycerides—to yield antimicrobial and antibiofilm properties.

Itis noted that in certain embodiments, extraction unit 210 and conversion unit 220 may be operated in reversed order, or possibly in parallel, so that the triglycerides are converted into monoglycerides, fatty acid salts and/or FFA of MCFAs already in BSF larvae 90 and prior to separation of (MCFAs-rich) BSFL oil as MBSFL oil 225.

In certain embodiments, conversion unit 220 may be configured to convert the triglycerides into monoglycerides, fatty acid salts and/or FFA of MCFAs by saponification and/or hydrolysis (e.g., acidic or enzymatic). Conversion unit 220 may be configured to break off fatty acids from triglyceride fats in BSFL oil 215 to form monoglycerides and/or free fatty acids and/or fatty acid salts that exert the antimicrobial activity.

For example, in certain embodiments, BSFL 90 may be grinded and blanched and/or heated, the pH of the resulting slurry may be adjusted, and the slurry may then be subjected to lipolytic enzymes such as lipase, to increase free lauric acid fractions (releasing lauric acid from the triglyceride complex). The slurry may then be heated to inactivate the enzymes and the slurry may be separated into its oil, protein and water fractions, e.g., using a decanter and/or a centrifuge. Alternatively, or complementarily, the lipase enzymes may be added after the separation stage, to the oil fraction and/or to the meal fraction separately. Alternatively, or complementarily, the slurry may be dried to produce whole fat insect meal with modified oil having increased antimicrobial properties. In certain embodiments, the remaining meal fraction may have high oil content (e.g., 10% or more) and may be further treated to yield additional laurate, monolaurin and/or free lauric acid-enriched products. In certain embodiments, lipolytic enzymes may be used to decompose the lipids, e.g., as taught by Japanese Patent Publication No. 2009254348 for processing bee larvae.

MBSFL oil 225 may be rich in monolaurin and free lauric acid and/or laurate, and as a result have antimicrobial properties for a wide range of applications, without requiring the digestion of BSFL oil with triglycerides by animals that is taught in the prior art.

In certain embodiments, system 200 may further comprise a separation unit 230 configured to separate MCFAs 235 from MBSFL oil 225 and optionally enrich MBSFL oil 225 with extracted MCFAs 235. For example, Norulaini et al. describes the separation of lauric acid and oleic acid in palm kernel oil using fractional supercritical carbon dioxide (SC—CO₂) extraction (Norulaini et al. 2004, Supercritical enhancement for separation of lauric acid and oleic acid in palm kernel oil (PKO); Separation and Purification Technology 39, 133-138). Alternatively, the glycerol fraction can be extracted out, leaving behind MCFAs rich fraction together with additional essential fatty acids. An example for purifying glycerol is described by H. W. Tan et al., using a distillation process (Tan et al. 2013, Glycerol production and its applications as a raw material: A review; Renewable and Sustainable Energy Reviews 27, 118-127). In certain embodiments, separation unit 230 may be configured to separate out glycerol from the MBSFL oil to yield MCFAs-rich MBSFL oil.

In certain embodiments, formulation 245 may be configured to suppress any of Gram-positive bacterial biofilms, Gram-negative bacterial biofilms, fungal biofilms and/or growth of microorganisms. In certain embodiments, formulation 245 may be configured to be applicable in at least one of: Dermal applications, topical therapy, oral applications, medical equipment and/or industrial applications (see examples below). In certain embodiments, formulation 245 may be configured to prepare the formulation as a dermal or oral crème, serum, wash, suspension and/or solution, or any other type of formulation. In certain embodiments, system 200 may further comprise an applicator 250, such as any appropriate device like a dispenser, collapsible container, sprayer etc., configured to apply formulation 245.

In certain embodiments, the conversion process may be configured to enhance BSFL oil dissolution in water, resulting in MBSFL oil with improved solubility with respect to BSFL oil. For example, low water solubility of a substance may sometimes be used to modify its relative antimicrobial inactivity (Griffin et al., 1999, The role of structure and molecular properties of terpenoids in determining their antimicrobial activity; Flavour and Fragrance Journal 14(5): 322-332). In addition, solubility could well be a limiting factor with respect to practical applications. Table 3 provides solubility data for BSFL oil before and after the saponification (conversion) process.

TABLE 3
Solubility data of BSFL oil, before and after conversion to
MBSFL oil. Test method: Food Chemicals Codex (FCC).

| Product | Solubility |
|---|---|
| BSFL oil (lot 0030.111217) | Not soluble |
| BSFL oil (lot 0095-0098.181210) | Not soluble |
| MBSFL oil (lot 0030.11.12.17-181125) | 99.93% |
| MBSFL oil (lot 0095-0098.181210-181227) | 99.68% |

The following data demonstrate, in a non-limiting manner, the application of the MBSFL oil to suppress biofilm development and/or microorganism growth. MBSFL oil (converted, 225, BioBee Sde Eliyahu Ltd, lot 0095-0098.181210-181227) was compared to BSFL oil (before conversion, 215, BioBee Sde Eliyahu Ltd, lot 0030.111217), lauric acid 98% (Sigma-Aldrich, denoted LA) and chlorhexidine acetate (Steinberg, denoted CHX), were analyzed as antimicrobial and anti-biofilm-formation agents in vitro, with respect to Pseudomonas aeruginosa (Gram negative, denoted PA01), Staphylococcus aureus (ATCC, American Type Culture Collection) (Gram positive, denoted SA), Streptococcus mutans (ATCC) (Gram positive, denoted SM), Lactobacillus (ATCC) (Gram positive, denoted L), Candida albicans (ATCC) (yeast, denoted CA) and Candida glabrata (ATCC) (yeast, denoted CG).

Microbial stocks of the Biofilm Research Laboratory were grown from frozen stock: SM at overnight 37° C. in BHI (brain heart infusion) broth medium in an atmosphere of 5% CO₂; L at 37° C. in MRS (de Man, Rogosa and Sharpe) broth medium in an atmosphere of 5% CO₂; PAO1 and SA at 37° C. in TSB (Trypticase soy broth) medium; CA and CG at 37° C. in RPMI (Roswell Park Memorial Institute) medium.

BSFL oil and MBSFL oil were prepared in DDW by heating at 100° C. LA was prepared at 50% Ethanol/50% DDW (doubly distillated water) and heated constantly to prevent solidifying in the tube. Maximum concentration of tested agents was the same (2.7% of lauric acid content). CHX was prepared in DDW

To examine the effects of MBSFL oil on microorganism's growth (planktonic conditions), serial 1:2 dilutions of each tested agent in appropriate medium (BSFL, MBSFL and LA from 2.7 to 0.04%; CHX from 20 μg/ml to 0.3 μg/ml) were prepared in a polystyrene flat-bottomed 96-well microplate. Wells with no compounds and those with bacteria served as positive controls. Wells with no bacteria and with compound served as blanks. An equal volume (100 μl) of the each tested bacterial and fungal suspension at optical density (OD)₅₉₅=0.02 and 0.05, respectively, was added to each well. After a 24 h incubation, growth was monitored by recording the OD at 595 nm using a Genius plate reader Spectrophotometer (Tecan). The assay was performed in triplicates.

To examine the effects of MBSFL oil on microorganism's viability, equal amounts (5 μl) of bacterial suspension exposed to each tested concentration of agent was plated on agar plates. Plates were incubated overnight at 37° C. Colony growth was recorded as non-affected by agent's viable bacteria. Minimal Bactericidal Concentration (MBC) was recorded as no colony growth. Minimal Fungicidal Concentration (MFC) was recorded as no fungal growth.

To examine the effects of MBSFL oil on biofilm formation by the microorganisms, assays for all of the tested agents were performed as described above except of growth medium used which was MRS/BHI supplemented with 1% sucrose and TSB/RPMI supplemented with 1% glucose. After incubation for 24 h, spent media and free-floating bacteria/fungi were removed by gentle aspiration and the wells were washed twice with phosphate-buffered saline (PBS, pH 7.4). The biofilm was then quantified by crystal violet staining. Briefly, 0.02% crystal violet was added into wells for 45 min, which were then washed twice with DDW to remove unbound dye. After adding 200 μl of 30% acetic acid into each well, the plate was shaken for 10 min to release the dye and the biofilm was quantified by measuring the absorbance at 595 nm using a Genius plate reader Spectrophotometer (Tecan). Assay was performed in triplicates.

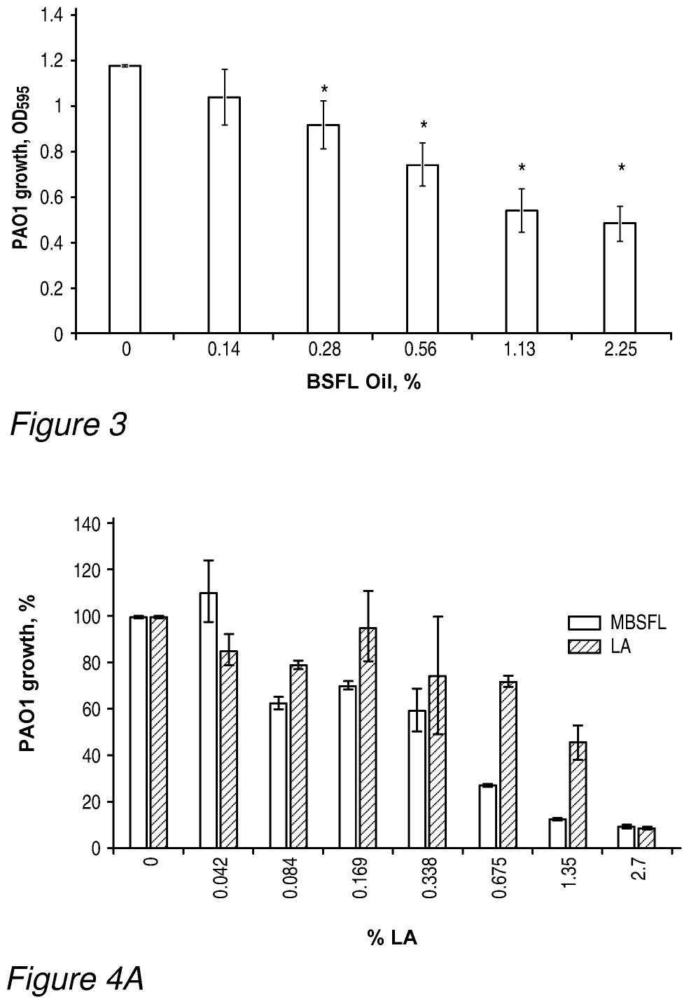

FIG. 3 provides results of the effect of BSFL oil on Pseudomonas aeruginosa (PAO1) growth in planktonic environment, according to some embodiments of the invention. The means and standard deviations of the bacterial growth are shown, and significant differences (from no BSFL oil, p<0.01) according to Student's t-test are denoted be asterisks (*). No minimum inhibitory concentration (MIC) was detected, however, PAO1 growth was reduced dose-dependently with increasing BSFL oil doses. BSFL oil at doses of 0.28%, 0.56%, 1.12% and 2.25% was able to decrease PAO1 growth by 23%, 37%, 55% and 60%, respectively.

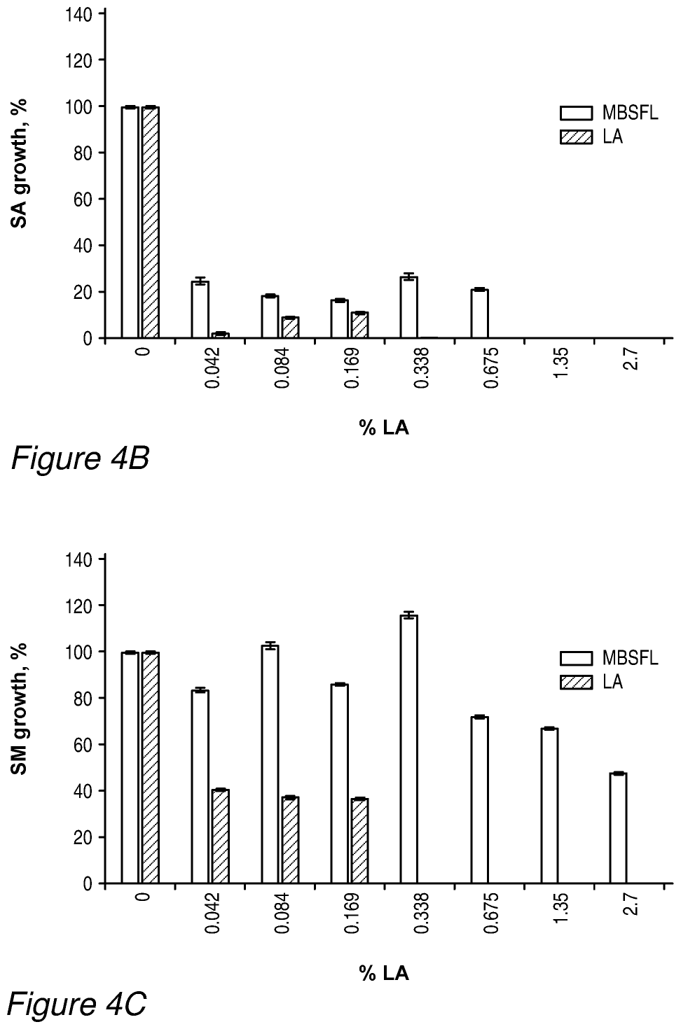

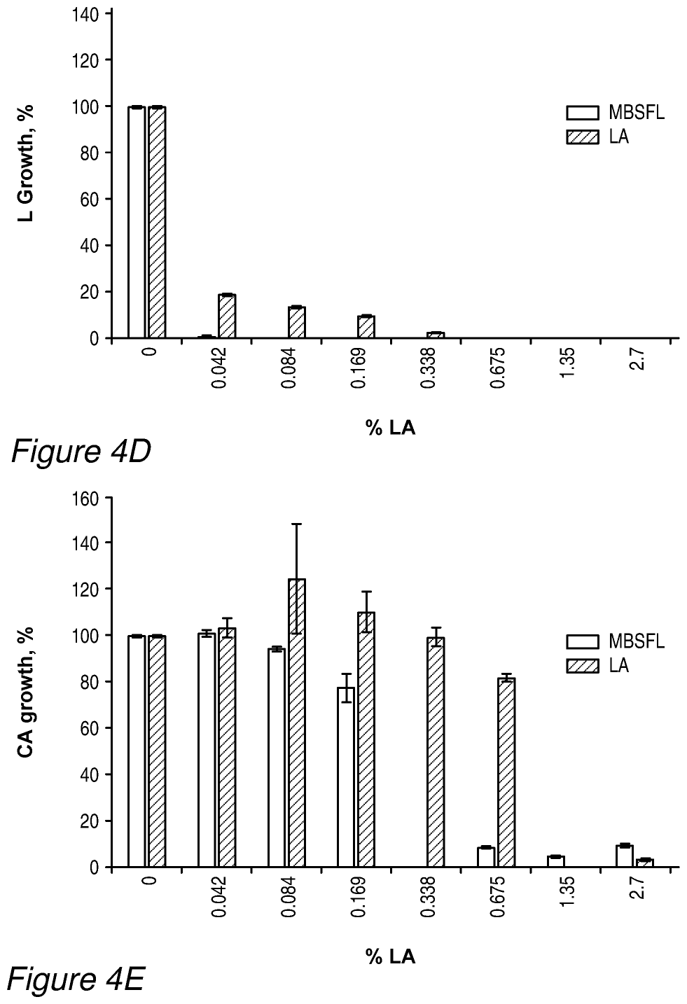

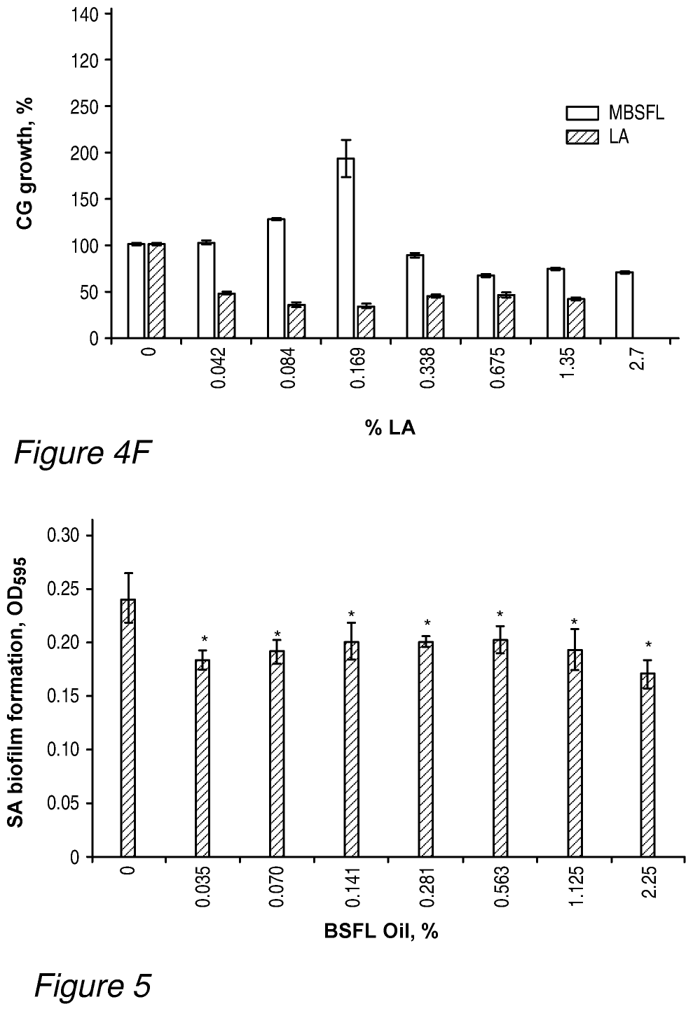

FIGS. 4A-F provide comparative results indicating the effect of MBSFL oil and lauric acid (LA) on the growth of various types of microorganisms in planktonic environment, specifically Pseudomonas aeruginosa (PAO1, FIG. 4A), Staphylococcus aureus (SA, FIG. 4B), Streptococcus mutans (SM, FIG. 4C), Lactobacillus (L, FIG. 4D), Candida albicans (CA, FIG. 4E) and Candida glabrata (CG, FIG. 4F), according to some embodiments of the invention. The effect of BSFL oil on microorganism's growth was compared to MBSFL oil and lauric acid 98%. The effect of BSFL oil was at a much lower degree in comparison with MBSFL oil, as MBSFL oil was able at dose of 1.35% to reduce more than 80% of PAO1 growth (FIG. 4A). Among all tested microbes, planktonic growth was notably affected by BSFL oil in regard to PAO1 only. As the effect of BSFL oil on the rest of the microorganisms tested was negligible, data are not shown. It is noted that in the presented experiments, gram-positive bacterial and fungal growth were inhibited by MBSFL oil and not by native BSFL oil. FIGS. 4B, 4C, 4D, 4E and 4F illustrate the effect of MBSFL oil in comparison with LA on planktonic growth for SA, SM, L, CA and CG, respectively. Both MBSFL oil and LA dramatically affected growth of SA:LA already at lowest tested dose of 0.04% reduced SA growth by 90%, while MBSFL in range of doses 0.04%-0.67% reduced bacterial growth by 75%, exhibiting MIC at dose of 1.35% (FIG. 4B). MBSFL oil moderately reduced SM growth, however it did not exhibit either MIC or MBC. In contrast, LA notably decreased bacterial growth and was able to exhibit MIC at dose of 0.33% (FIG. 4C). Both MBSFL oil and LA dramatically affected L growth: MICs for LA and MBSFL were 0.33% and less than 0.04%, respectively (FIG. 4D). Both MBSFL and LA notably reduced fungal growth: MBSFL oil demonstrated inhibition of CA growth, with MIC at dose of 0.33%. LA was less potential than MBSFL oil by exhibiting MIC at dose of 1.35% (FIG. 4E). For CG, MBSFL oil demonstrated moderate inhibition of CG growth, with non-detectable MIC. LA was more potential by exhibiting MIC at dose of 2.7% (FIG. 4F). These data altogether, indicate that conversion 120 significantly increased the anti-microbial effects of the BSFL oil.

Although MBSFL oil results show growth inhibition effect, no MBC/MFC was detected for MBSFL oil at all tested doses for all of the microorganisms tested: PAO1, SA, SM, CA and CG. Only exception is with L, for which MBC for MBSFL oil was less than 0.08%. Whereas MBC/MFC for MBSFL oil was detected for one microbe only, LA exerted MBC/MFC for all tested microbes, except PAO1:MBC for SA was detected at dose of 0.67%, MBC for SM was detected at dose of 0.33%, MBC for L was detected at dose of 0.67%, MFC for both CA and CG was detected at dose of 2.7%.

FIG. 5 provides comparative results indicating the effect of BSFL oil on biofilm formation of Staphylococcus aureus (SA), according to some embodiments of the invention. Unmodified BSFL oil caused insignificant effect on biofilm formation, affecting only SA by a moderate decrease of biofilm formation in the range of 20%-29%. As biofilm formation of the rest of microbes was not affected by BSFL oil, data are not shown.

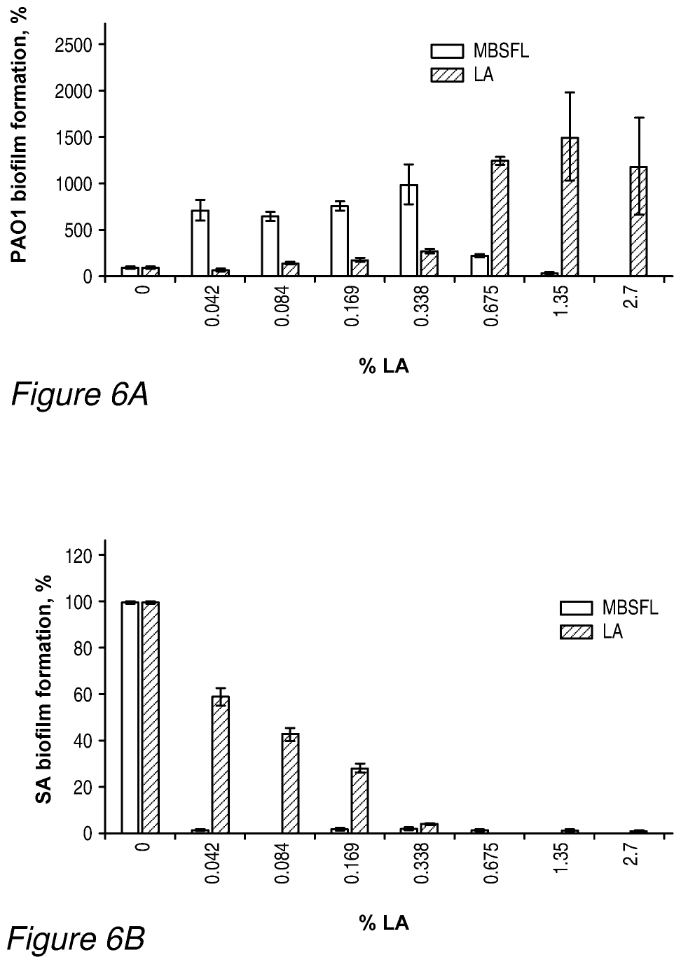

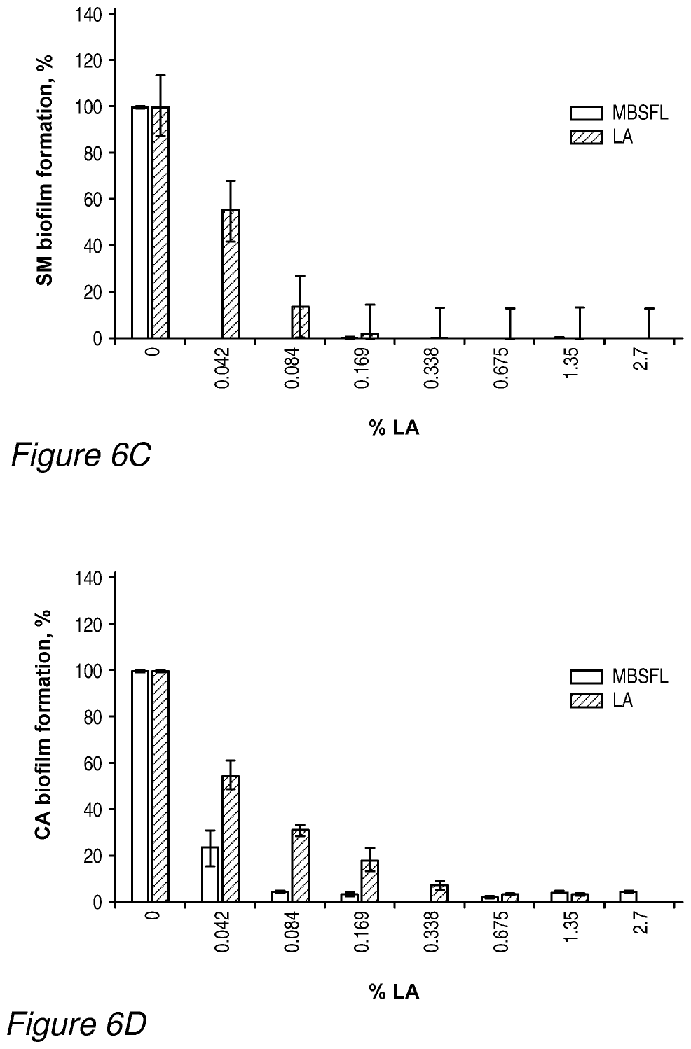

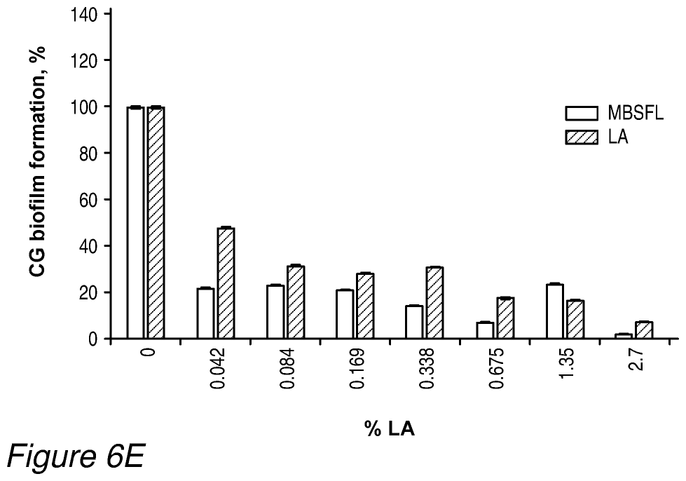

FIGS. 6A-E provide comparative results indicating the effect of MBSFL oil and lauric acid (LA) on biofilm formation of various types of microorganisms: Pseudomonas aeruginosa (PAO1, FIG. 6A), Staphylococcus aureus (SA, FIG. 6B), Streptococcus mutans (SM, FIG. 6C), Candida albicans (CA, FIG. 6D) and Candida glabrata (CG, FIG. 6E), according to some embodiments of the invention. FIGS. 6A-6E illustrate that the modification of BSFL oil increased its antibiofilm properties. In addition, MBSFL oil demonstrated a much more pronounced effect on biofilm formation of all tested microbes as compared to LA:MBSFL oil at doses of up to 0.33% increased biomass of PAO1, while increasing MBSFL oil doses starting from 0.67% inhibition of biofilm formation. MBSFL oil at doses of 1.35% and 2.7% almost totally inhibited biofilm formation. In contrast, LA dose-dependently increased biomass at all concentrations tested (FIG. 6A). Biofilm formation of SA was dramatically inhibited by MBSFL oil already at lowest tested dose of 0.04% (almost no biofilm was formed), while LA was able dose-dependently to decrease biofilm formation and at 0.67% was totally inhibited biofilm formation (FIG. 6B). MBSFL oil and LA also exerted obvious anti-biofilm effect against SM:LA was able to totally inhibit biofilm formation at dose of 0.16%, while MBSFL oil was even more effective demonstrating total biofilm inhibition at dose lower than the lowest tested dose (FIG. 6C). Biofilm formation of CA was strongly affected by both MBSFL oil and LA, with a stronger inhibition effect of MBSFL oil compared to LA:MBSFL oil was able almost totally to inhibit biofilm formation of CA at dose of 0.08%, in comparison with dose of 0.67% for LA (FIG. 6D). Biofilm formation of CG was inhibited by MBSFL oil by 80% already at lowest tested dose of 0.04%. LA was also effective against biofilm formation of CG but with less impact (FIG. 6E). Since biofilm of L was very weak and easily washed out, no data were demonstrated on biofilm formation.

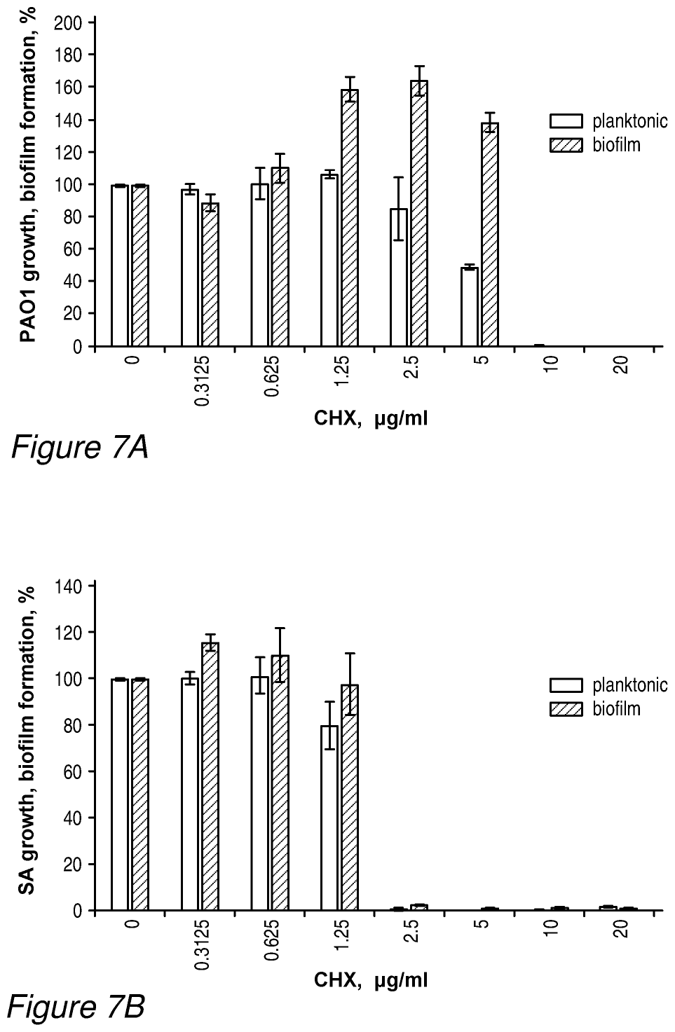

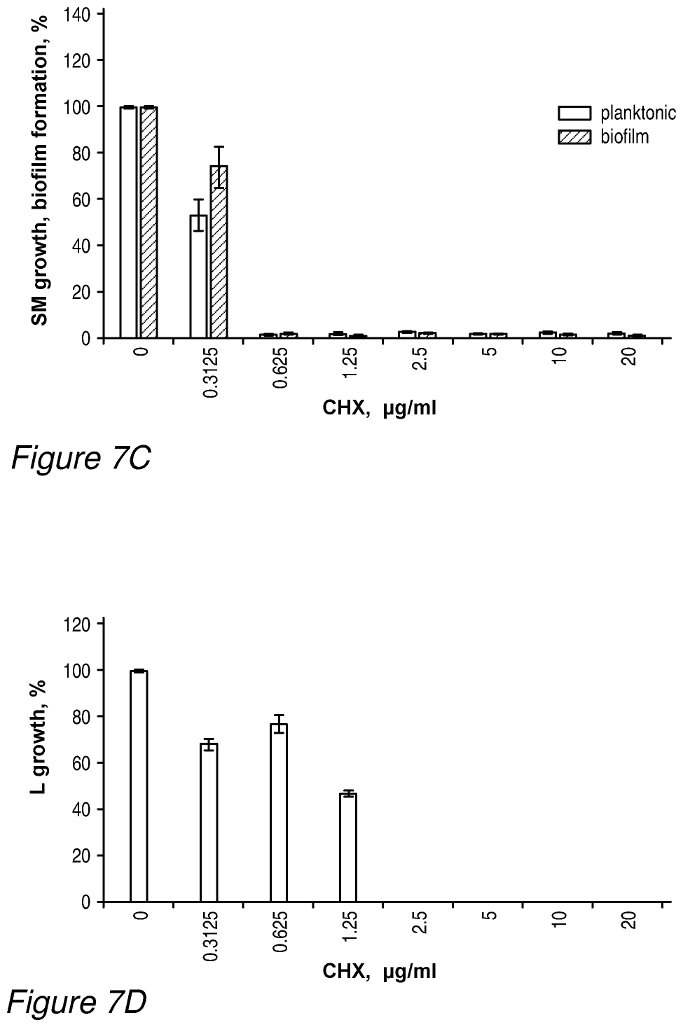

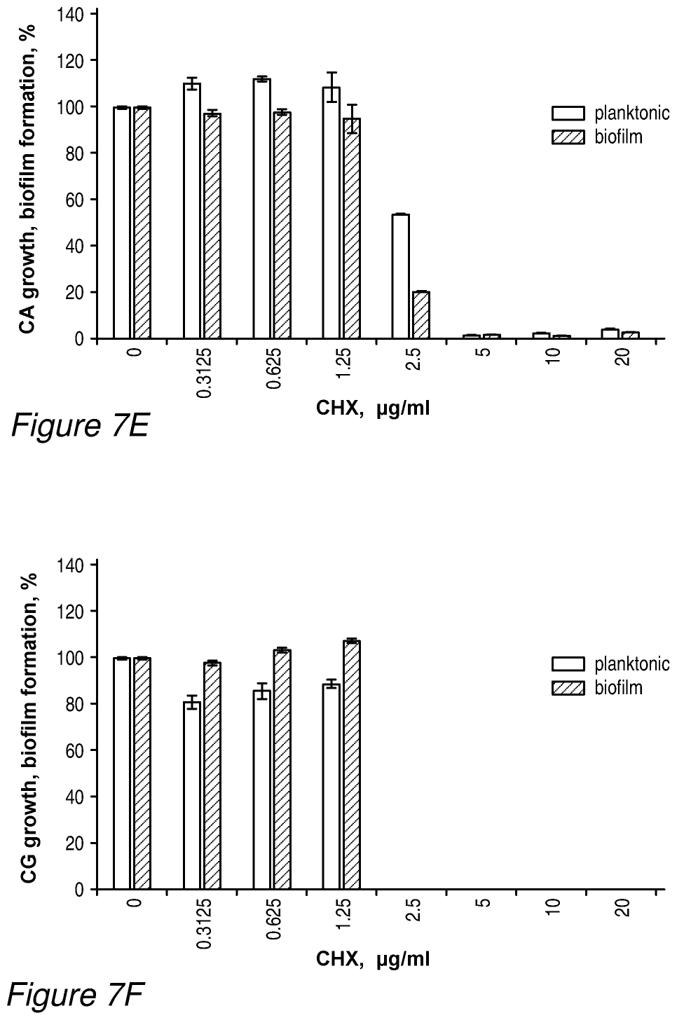

FIGS. 7A-F provide comparative results indicating the effect of chlorhexidine acetate (CHX) on planktonic growth and biofilm formation of various types of microorganisms: Pseudomonas aeruginosa (PAO1, FIG. 7A), Staphylococcus aureus (SA, FIG. 7B), Streptococcus mutans (SM, FIG. 7C), Lactobacillus (L, FIG. 7D), Candida albicans (CA, FIG. 7E) and Candida glabrata (CG, FIG. 7F), according to some embodiments of the invention. The positive control agent, CHX, exhibited total inhibition of PAO1 growth and biofilm formation at the same dose of 10 μg/ml (FIG. 7A), MBC was detected at dose of 20 μg/ml. CHX inhibited growth and biofilm formation of SA at the same dose of 2.5 μg/ml (FIG. 7B), MBC was detected at dose of 10 μg/ml. CHX inhibited growth, biofilm formation and exhibited MBC against SM at the same dose of 0.625 μg/ml (FIG. 7C). MIC and MBC for CHX against L were detected at the same dose of 2.5 μg/ml (FIG. 7D). Since biofilm of L was very weak and easily washed out, no data were demonstrated on biofilm formation. CHX inhibited growth and biofilm formation of CA and CG and exhibited MFC at the same dose of 5 μg/ml and 2.5 μg/ml (FIGS. 7E and 7F, respectively). CHX serves as a positive control, demonstrating a non-specific killing effect.

The following data demonstrate, in a non-limiting manner, the antifungal activity of MBSFL oil against the following fungi: Trichophyton rubrum (denoted T. rubrum), Microsporum canis (denoted M. canis) and Epidermophyton floccosum (denoted E. floccosum). MBSFL oil (converted, 225, BioBee Sde Eliyahu Ltd, lot 0095-0098.181210-181227) was tested in vitro in comparison with the antifungal drugs Bifonazole and Terbinafine.

MBSFL oil was prepared in DDW at a 1:1 ratio by heating and stirring at 100° C. Bifonazole and Terbinafine were used as is.

Fungi strains (SB Clinic research center, mycological laboratory, Israel) were taken and inoculated in Sabouraud dextrose agar broth at 33±1° C.

To examine the effect of MBSFL oil on fungi growth, Sabouraud Dextrose Agar (SDA) was heated to liquification and then each reagent was suspended in SDA grown medium in the following ratio: 13% MBSFL oil (Group B), 13% Bifonazole (group C) and 20% Terbinafine (group D). Group A of plates with SDA agar grown medium with no reagent addition served as control. Finally, the grown medium of each group was poured to agar plates (2 ml plate), and allowed to dry. When the petri dishes had dried, 1.5×1.5 cm of each fungus was placed in the center of each petri dish. Plates were incubated at 35±2° C. for 22 days. The plates were then taken out after 7, 14, 18 and 22 days and on each day the fungi diameter was measured using a scale.

Tables 4A and 4B provide experimental results indicating the antifungal efficiency of MBSFL oil, according to some embodiments of the invention. Table 4A provides comparative results indicating the effect of MBSFL oil, Bifonazole and Terbinafine on the growth of T. rubrum, M. canis and E. floccosum. MBSFL oil exhibited full growth inhibition against all fungi tested, similar to Bifonazole and Terbinafine at the specific concentration tested. Table 4B provides experimental results comparing MBSFL oil to Terbinafine hydrochloride 1% % w/w in spray topical formulation (Lamisil®) in their efficiency to inhibit Trichophyton rubrum growth. Fungal growth was monitored over two weeks in petri dishes containing dissolved SDA (Sabouraud Dextrose Agar) substrate with T. rubrum samples.

TABLE 4A
The effect of antifungal agents against T. rubrum, M. canis and E. floccosum growth vs. time.

| | | Diameter | Diameter | Diameter | Diameter | Diameter |
|---|---|---|---|---|---|---|
| Group, | | (cm), | (cm), | (cm), | (cm), | (cm), |
| Reagent | Fungi | t = 0 | t = 7 d | t = 14 d | t = 18 d | t = 22 d |
| A, None | T. rubrum | 1.5 × 1.5 | 1.5 × 1.5 | 3.0 × 3.0 | 6.0 × 6.0 | full growth |
| | M. canis | 1.5 × 1.5 | 1.5 × 1.5 | 3.0 × 3.0 | 6.0 × 6.0 | full growth |
| | E. flucosum | 1.5 × 1.5 | 1.5 × 1.5 | 3.0 × 3.0 | 6.0 × 6.0 | full growth |
| B, MBSFL oil | T. rubrum | 1.5 × 1.5 | 1.5 × 1.5 | 1.5 × 1.5 | 1.5 × 1.5 | 1.5 × 1.5 |
| | M. canis | 1.5 × 1.5 | 1.5 × 1.5 | 1.5 × 1.5 | 1.5 × 1.5 | 1.5 × 1.5 |
| | E. flucosum | 1.5 × 1.5 | 1.5 × 1.5 | 1.5 × 1.5 | 1.5 × 1.5 | 1.5 × 1.5 |
| C, Bifonazole | T. rubrum | 1.5 × 1.5 | 1.5 × 1.5 | 1.5 × 1.5 | 1.5 × 1.5 | 1.5 × 1.5 |
| | M. canis | 1.5 × 1.5 | 1.5 × 1.5 | 1.5 × 1.5 | 1.5 × 1.5 | 1.5 × 1.5 |
| | E. flucosum | 1.5 × 1.5 | 1.5 × 1.5 | 1.5 × 1.5 | 1.5 × 1.5 | 1.5 × 1.5 |
| D, Terbinafine | T. rubrum | 1.5 × 1.5 | 1.5 × 1.5 | 1.5 × 1.5 | 1.5 × 1.5 | 1.5 × 1.5 |
| | M. canis | 1.5 × 1.5 | 1.5 × 1.5 | 1.5 × 1.5 | 1.5 × 1.5 | 1.5 × 1.5 |
| | E. flucosum | 1.5 × 1.5 | 1.5 × 1.5 | 1.5 × 1.5 | 1.5 × 1.5 | 1.5 × 1.5 |

TABLE 4B
Experimental results comparing MBSFL oil to Terbinafine hydrochloride 1%
(Lamisil ®) asinhibitors of T. rubrum growth.

| Experiment | Implantation | Week 1 | Week 2 | Growth (%) |
|---|---|---|---|---|
| Control | 0.8 × 1.0 | 2.0 × 1.7 | 4.3 × 3.0 | 1612% |
| | 0.4 × 1.0 | 1.7 × 2.1 | 3.5 × 4.8 | 4200% |
| | 0.8 × 0.5 | 1.6 × 1.7 | 3.5 × 3.5 | 3062% |
| MBSFL 1% | 1.1 × 0.7 | 4.0 × 3.5 | 4.0 × 4.0 | 2078% |
| | 0.7 × 0.6 | 4.0 × 3.7 | 5.0 × 4.0 | 4762% |
| | 0.8 × 0.5 | 1.8 × 1.8 | 4.0 × 4.0 | 4000% |
| MBSFL 4% | 1.0 × 0.6 | 0.6 × 1.0 | 1.0 × 0.6 | 0% |
| | 0.8 × 0.8 | 0.8 × 0.8 | 0.8 × 0.8 | 0% |
| | 0.9 × 0.7 | 0.7 × 0.9 | 0.9 × 0.7 | 0% |
| Lamisil ® | 1.0 × 1.0 | 1.0 × 1.0 | 1.0 × 1.0 | 0% |
| | 1.0 × 1.0 | 1.0 × 1.0 | 1.0 × 1.0 | 0% |
| | 0.7 × 0.7 | 0.7 × 0.7 | 0.7 × 0.7 | 0% |

The results indicate that MBSFL oil at 4% concentration inhibits T. rubrum growth in vitro as good as the known antifungal agent Lamisil®, indicating it being a potent anti-fungal agent. The anti-fungal activity of MBSFL oil in this experiments was dose dependent, with low concentrations (I1%) not being active. As Lamisil® is used to treat tinea pedis (athlete's foot) and tinea cruris (dhobie itch/jock itch) caused by Trichophyton (e.g., T. rubrum, T. mentagrophytes, T. verrucosum, T. violaceum) and Epidermophyton floccosum, there are good grounds to suggest that MBSFL oil is likewise efficient in treating tinea pedis and related fungal infections in dermatological and non-dermatological diseases.

It is noted that the concentration of the MBSFL oil for inhibiting growth of micro-organisms is not limited to the values shown in the experiments above. Specifically, the required MBSFL oil concentrations depend on the composition of specific types of MBSFL oil, on the type(s) of micro-organism(s) of which growth is to be inhibited and on the conditions of the application. For example, the MBSFL oil was shown in preliminary experiments to inhibit growth of Candida albicans at a concentration of about 0.3%. Accordingly, the concentration of the MBSFL oil may be any of at least 0.1%, 0.3%, 0.5%, 1%, 2%, 3%, 5%, 10% or any intermediate value or range of values.

In certain embodiments, MBSFL oil may be used to inhibit viral growth, as disclosed above. For example, viruses such as Porcine Epidemic Diarrhea Virus (PEDV), fat-enveloped viruses (e.g., Marek, Newcastle disease (ND), infectious bronchitis (IB), avian influenza (AI)), Vesicular stomatitis virus (VSV) strain Indiana, Herpes simplex virus type 1 (HSV-1) strain MacIntyre, Visna Virus (VV), Junin virus (JUNV), HIV virus and possibly Covid 19/nCoV-2019 were shown to be inhibited by lauric acid, monolaurin, medium-chain fatty acids (MCFA) and/or coconut oil, indicating the potential anti-viral efficiency of disclosed MBSFL oil.

It is suggested, without being bound by theory, that antiviral fatty acids (and especially their monoglycerides) may cause leakage of the viral envelope, and at higher concentrations, may cause a complete disintegration of the envelope and of the viral particles, and possibly also disintegration of the plasma membranes of tissue culture cells resulting in cell lysis and death. The fatty acid concentration required for maximum viral inactivation may vary and can be determined experimentally, with respect to the type of MBSFL oil and details of the application.

To summarize, the data show that the modification of the BSFL oil by converting triglycerides to monoglycerides, fatty acid salts and/or free fatty acids (FFA) of lauric acid notably enhances its antimicrobial properties, both in planktonic and biofilm environment. In addition, the MBSFL oil's effect on bacterial and fungal growth was similar to that of LA. However, MBSFL oil demonstrated a much more profound effect on biofilm formation for all tested microbes as compared to LA. Moreover, MBC/MFC for MBSFL oil was detected for one microbe only. In contrast, LA exerted MBC/MFC for all tested microbes, except PAO1. Antibacterial agents are usually regarded as bactericidal if the MBC is no more than four times the MIC. Due to its strong inhibitory effect on biofilm formation with no bactericidal effect, which may reflect a unique and specific activity, MBSFL oil may be used as a specific anti-biofilm compound that is unique and applicable in many clinical applications and may be applied to additional pathogens and possibly in a sustained release delivery system.

Non-limiting examples for possible applications of converted BSFL meal or oil, and/or lauric acid derived therefrom and/or formulations containing either preparations comprise a range of antibacterial, anti-viral and anti-fungaluses, against planktonic microorganisms and/or as anti-biofilm agents. For example, dentistry applications may comprise oral cavity applications such as treatment of thrush, stomatitis and orthodontal issues or presentation of caries and plaque induced gingivitis or periodontitis. For example, formulations 245 may comprise mouthwash, toothpaste, either as adjuvant or as stand-alone formulations. In additional examples, applications may comprise dermal applications such as: (i) Atopic dermatitis, psoriasis, contact dermatitis and/or diaper rash—BSFL/MBSFL oil may affect the dryness of the skin by its moisturizing properties and in addition, MBSFL oil may prevent the development of secondary infections, such as by S. aureus, which are known to develop in these diseases following patient scratching of the skin area, due to the itching symptom; (ii) Tinea pedis (fungal infection of legs)—BSFL/MBSFL oil may affect the fungal infection of the skin by its antifungal properties, and in addition, MBSFL oil may prevent the development of secondary bacterial infections, which are known to be developed in these diseases following patient scratching of the skin area, due to the itching symptom. Additional indications of the same family are scalp dandruff or seborrheic dermatitis, Pityriasis versicolor, cutaneous candidiasis, Tinea corporis and Tinea cruris. In various embodiments, formulation 245 may be used to treat (iii) Dermatophytosis—MBSFL oil may be useful against Trichophyton rubrum fungi (T. rubrum is a dermatophyte fungus responsible for a fungal infection of the skin known as dermatophytosis), and/or (iv) Onychomycosis (Tinea unguium, nail fungal infection)—MBSFL oil may be useful for this and other dermatology indications. In various embodiments, formulation 245 may be used to treat (v) acne vulgaris—In vitro studies (Nakatsuji et al. 2009, Antimicrobial property of lauric acid against Propionibacterium acnes: its therapeutic potential for inflammatory acne vulgaris, J Invest Dermatology 129(10): 2480-2488) indicate antimicrobial properties of lauric acid against Propionibacterium acnes and highlight the potential of using lauric acid as an alternative treatment for antibiotic therapy of acne vulgaris. In various embodiments, formulation 245 may be used to treat (vi) urinal tract infections. In various embodiments, formulation 245 may be used to treat (vii) vaginal infections—MBSFL oil may be useful for treating candida (as was indicated for both Candida albicans and Candida glabrata) and bacterial vaginosis due to its antibacterial and antifungal effects, e.g., for treatment of vaginal dryness and for avoiding irritation, e.g., with formulation 245 used as intimal wash and/or vaginal capsules. In various embodiments, formulation 245 may be used to treat (viii) ear infection (otitis externa, acute otitis media) and/or (ix) Xerosis and/or providing anti-aging effects on skin—MBSFL oil may be useful for providing moisturizing effects, inhibition of skin water loss and enhancing of skin regeneration capability effects of lauric acid and linoleic acid. In various embodiments, formulation 245 may be used to treat (x) Diabetic foot ulcers (DFU), being one of the most severe complications in Diabetes mellitus. Ischemic and neuropathic lesions are of major importance for DFU onset; however, it is the infection by multidrug-resistant and biofilm-producing microorganisms, along with local microenvironmental conditions unfavorable to antibiotics action that ultimately cause infection chronicity and lower limbs amputation. MBSFL may be useful for treating DFU as it exerts a strong inhibitory effect on biofilm formation of both S. aureus and P. aeruginosa which are the predominant Gram-positive and Gram-negative species present in DFU, respectively (Santos et al. 2016, Guar gum as a new antimicrobial peptide delivery system against diabetic foot ulcers Staphylococcus aureus isolates, Journal of Medical Microbiology 65, 1092-1099). In various embodiments, formulation 245 may be used to treat (xi) Infections on the skin caused by antibiotic resistant bacteria. For example, MBSFL may be useful for treating Methicillin-resistant S. aureus (MRSA), a bacterium that causes infections in different parts of the body and is resistant to penicillin. In various embodiments, formulation 245 may be used to treat (xii) oral candidiasis (caused by Candida albicans accumulation). In various embodiments, formulation 245 may be used in (xiii) additional applications for local treatment with various types of viral infection related to Herpes: Herpes labialis, Herpes zoster and/or genital herpes. In various embodiments, formulation 245 may be used to treat (xiv) autoimmune diseases causing thickened skin such as Scleroderma and Ichthyosis. In various embodiments, formulation 245 may be used to treat (xv) various types of radiation burns, for example sunburn. Additional applications may comprise anti-biofilm coatings of catheters, implant devices, prostheses, mechanical valves, ventricular shunts, pacemakers, defibrillator, ventricular-assisted devices, Intervertebral disc and/or contact lenses to prevent device-associated infections of biofilm. Additional applications are industrial, such as anti-biofilm coatings on boats and/or pipes, cleaning agents for surfaces (e.g., tables) that remove bacterial infections (e.g., for hospitals, labs, kitchens, etc.). Additional examples for MBSFL oil applications may comprise veterinary applications as adjuvant in vaccines (fish, poultry) and topical applications in cats and dogs to promote the healing of cuts, wounds, hot spots, dry skin and hair, bites and stings, itching areas, as well as to prevent and treat yeast and fungal infections, including candida and possibly to reduce or eliminate bad breath in dogs

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A method comprising:
extracting black soldier fly larvae (BSFL) oil by processing BSFL,
modifying the BSFL oil into modified BSFL (MBSFL) oil by converting triglycerides in the BSFL oil to monoglycerides, fatty acid salts and/or free fatty acids (FFA) of medium chain fatty acids (MCFAs), and
applying the MBSFL oil to suppress biofilm development and/or microorganism growth.

2. The method of claim 1, further comprising extracting the MCFAs from the MBSFL oil and applying the extracted MCFAs to suppress biofilm development and/or microorganism growth.

3. The method of claim 1, further comprising enriching the MBSFL oil with MCFAs.

4. The method of claim 1, wherein the converting is carried out by saponification and/or hydrolysis.

5. The method of claim 1, wherein the converting is carried out to enhance a solubility of the converted MBSFL oil with respect to the BSFL oil.

6. The method of claim 1, wherein the applying is carried out against at least one of: Gram-positive bacterial biofilms, Gram-negative bacterial biofilm, fungal biofilm and growth of microorganisms and/or in at least one of: a dermal application and an oral application.

7. (canceled)

8. The method of claim 1, wherein the applying is carried out to treat at least one of: Atopic dermatitis, psoriasis, contact dermatitis, diaper rash, Tinea pedis, Dermatophytosis, Onychomycosis, Acne vulgaris, urinal tract infections, vaginal infections, ear infection, Xerosis, aging effects on the skin, diabetic foot ulcers, infections on the skin caused by antibiotic resistant bacteria, oral candidiasis, viral infection related to Herpes, autoimmune diseases, radiation burns, and topical therapy.

9. The method of claim 1, wherein the applying is carried out on at least one of: medical equipment and catheters: in an industrial application, pipes and ship's ballast water pipework; and/or in veterinary applications comprising at least one of: vaccine adjuvant for fish and/or poultry, topical applications in cats and dogs to promote the healing of cuts, wounds, hot spots, dry skin and hair, bites and stings, itching areas, as well as to prevent and treat yeast and fungal infections, including candida and/or to reduce or eliminate bad breath in dogs.

10. (canceled)

11. (canceled)

12. The method of claim 1, wherein the MCFAs comprise lauric acid in the form of laurate, monolaurin and/or free lauric acid.

13. A topical dermal or oral composition, comprising modified black soldier fly larvae (MBSFL) oil, modified from BSFL oil extracted from processed BSF larvae and comprising monoglycerides and/or fatty acid salts and/or free fatty acids of medium chain fatty acids (MCFAs) converted from triglycerides in the BSFL oil.

14. The topical dermal or oral composition of claim 13, wherein the MCFAs comprise lauric acid in the form of laurate monolaurin and/or free lauric acid.

15. A system comprising:
an extraction unit configured to extract black soldier fly larvae (BSFL) oil from BSFL,
a conversion unit configured to convert triglycerides in the prepared BSFL oil into monoglycerides, fatty acid salts and/or free fatty acids of medium chain fatty acids (MCFAs)—to yield modified BSFL (MBSFL) oil, and
a formulation unit configured to prepare from the MBSFL oil a formulation that suppresses biofilm development and/or microorganism growth.

16. The system of claim 15, wherein the conversion unit is configured to convert the triglycerides into the monoglycerides and/or fatty acid salts and/or free fatty acids of MCFAs by saponification and/or hydrolysis.

17. The system of claim 15, wherein the conversion unit is configured to enhance a solubility of the converted MBSFL oil with respect to the BSFL oil.

18. The system of claim 15, further comprising a separation unit configured to extract the MCFAs from the MBSFL oil and enrich the MBSFL oil with the extracted MCFAs; and/or configured to separate out glycerol from the MBSFL oil to yield MCFAs-rich MBSFL oil.

19. (canceled)

20. The system of claim 15, wherein the formulation is configured to suppress any of: Gram-positive bacterial biofilms and/or bacterial growth, Gram-negative bacterial biofilms and/or bacterial growth, fungal biofilms, fungal growth and/or growth of micro-organisms and/or be applicable in at least one of: dermal applications, oral applications, topical therapy, medical equipment, catheters, industrial applications.

21. The system of claim 15, wherein the formulation unit is configured to prepare the formulation as a dermal or oral crème, serum, wash, suspension and/or solution.

22. (canceled)

23. The system of claim 15, wherein the MCFAs comprise lauric acid in the form of laurate, monolaurin and/or free lauric acid.

24. The system of claim 23, wherein the MCFAs further comprises at least one of: capric acid (C10:0) in the form of capric acid salt (decanoate), monocaprin and/or free capric acid.

25. The system of claim 24, wherein the MBSFL oil comprises a combination of MCFAs lauric acid (C12:0) and capric acid (C10:0) with optional antimicrobial fatty acids myristic acid (C14:0), palmitoleic acid (C16:1), oleic acid (C18:1), linoleic acid (C18:2) and/or alpha linolenic acid (C18:3), converted by saponification and/or hydrolysis from the respective fatty acid triglycerides—to yield antimicrobial and antibiofilm properties.
